(19) United States
(12) Patent Application Publication
(10) Pub. No.: US 2007/0118153 A1
(43) Pub. Date: May 24, 2007

(54) EXTRACTION DEVICE AND MEDICAL SUTURING DEVICE SET

(75) Inventors: Shigeaki Funamura, Fukuroi (JP); Hitoshi Mabuchi, Fukuroi (JP)

Correspondence Address: TYCO HEALTHCARE - EDWARD S. JARMOLOWICZ, 15 HAMPSHIRE STREET, MANSFIELD, MA, 02048, US

(73) Assignees: Sherwood Services AG, Schaffhausen (CH); Nippon Sherwood Medical Industries Ltd., Fukuroi (JP)

(21) Appl. No.: 11/466,474

(22) Filed: Aug. 23, 2006

(30) Foreign Application Priority Data
Aug. 23, 2005 (JP) 2005-241131

(51) Int. Cl. A61B 17/04

(52) U.S. Cl. 606/148

(57) ABSTRACT

The extraction device is furnished with a pulling out device on which are formed hook elements that hook onto part of suture, which is disposed in an annular shape within the organism, and are able to draw this out of the organism. It is also a medical suturing device set that is used to anchor the organ to the organism's paries, which is furnished with a puncture needle, in which insertion through-hole is formed from the basal tip side to the apical tip side, and which is able to puncture the paries from the outside such that the aperture of insertion through-hole on the apical tip side is positioned within the organ; a suture that is supplied to the inside of the organ from the aperture via insertion through-hole, and which is able to from an annular section within the organ.

The present invention pertains to an extraction device and medical suturing device set used when anchoring human organs and paries.

Generally, when a catheter is inserted transdermally into the stomach, gallbladder, or other organs, a problem may arise in that the organ separates from the paries such that the catheter cannot be inserted into the organ. Therefore, it is necessary to anchor the organ and paries prior to catheter insertion and prevent the organ from separating from the paries.

Recently, the mainstream of medical suturing device sets has become a device in which two needles are used to hand over the suture within an organ, and knot both ends of the suture drawn out of the organism, thus anchoring the organ and paries. Such a device comprises a suture, a suture insertion needle capable of inserting a suture into an organ, and an organ anchoring needle equipped with a tubular external needle and an internal needle having a tip equipped with a snare loop inserted into and capable of sliding within the external needle; by means of the snare loop that bends axially with respect to the internal needle and that protrudes from the tip aperture of the external needle pursuant to the sliding motion of the internal needle, one end of the suture protruding from the suture insertion needle is supported and drawn out from the organism (for example, refer to Japanese Patent Application No. JP 5-161655 (1993), in particular FIG. 4).

In a conventional medical suturing device set, two needles are used to hand over the suture, so when the relative positions of the needle tips within the organ after puncturing are not correct, or when there is insufficient space within the organ and the snare loop is deformed against the organ wall, it becomes difficult for the suture to be supported by the snare loop, so its use is limited to experienced health care personnel.

### SUMMARY OF THE INVENTION

The object of the present invention is to offer an extraction device and medical suturing device set that solves the aforementioned problems by readily handing over the suture, so that it can be handled even by inexperienced health care personnel.

The invented extraction device is an extraction device used to extract a suture positioned within an organism to the outside of the organism, characterized by the fact that this is furnished with an extraction needle in which an engagement member is formed capable of catching onto that part of the suture positioned circularly within the organism is looped, and drawing this outside of the organism.

In the present invention, the suture inserted into the organism is engaged by the engagement member of the extraction needle, so it can be drawn out of the organism at a place that is different from the insertion location. Because this makes it easy to hand over the suture, it can be handled even by inexperienced health care personnel.

In one invented extraction device, the extraction device is furnished with a mantle that forms a cavity that permits the extraction needle to pass entirely through so as to be exposed from the tip. In the present invention, it is possible to insert the extraction needle into the cavity of the mantle, thus making it possible to reduce the invasiveness to the body when drawing the extraction needle on which the suture is caught outside of the body.

In one invented extraction device, the extraction device is furnished with: an extraction puncture needle formed with an internal cavity from the basal tip side through the apical tip side; and a pulling out device in which is formed an engagement member that can be inserted through the internal cavity so as to be exposed from the apical tip side of the extraction puncture needle, such that the portion that is exposed from the apical tip side of the extraction puncture needle engages part of the suture that has been positioned in a circular shape within the organism and draws it out from the organism.

In the present invention, it is possible to engage the suture that has been inserted into the organism by the engagement member of the extraction device, and draw it out of the organism, though the cavity of the extraction puncture needle, at a location that is different from the insertion location. This makes it easy to hand over the suture, so it can be handled even by inexperienced health care personnel.

In one invented extraction device, the engagement member is structured by a series of multiple hook elements; the hook elements are formed with a base that can support part of the suture, a primary extension that is connected to part of the base and extends towards the basal tip of the pulling out device, a secondary extension that is connected to the other part of the base and that extends to the basal tip of the pulling out device, and a gap that is formed so as to envelope the periphery of the suture by the base and the primary extension and the secondary extension, and the series of hook elements is structured so that the primary extension of a hook element is connected to the secondary extension of another hook element that is adjacent to the hook element.

In the present invention, it is possible to have a structure in which the hook elements are facing several different directions outwards, towards 2 outer directions, or towards 4 outer directions; there is no limitation of the peripheral positioning of hook elements, which thus makes it easy to engage the suture that has been disposed in an annular form.

In one invented medical suturing device set, a medical suturing device set that is used to anchor the organ of an organism to the paries of the aforementioned organism is furnished with: an insertion puncture needle, in which is formed an insertion through-hole from the basal tip side through the apical tip side, and that is able to puncture the paries from the outside such that the aperture of the insertion through-hole on the apical tip side is positioned within the organ; a suture that, passing through the insertion through-hole, is supplied within the organ from the aperture, and that is able to form a circular section within the organ; and any one of the aforementioned extraction devices that is able to engage part of the circular section of the suture and draw it out of the organism.

In the present invention, provision is made for an extraction device, an insertion puncture needle, and a suture, which makes it easy to hand over the suture, so the desired organ and paries can be securely anchored with low invasiveness even by inexperienced health care personnel. It is also possible to draw out a single suture from multiple locations, thus, by taking this together and ligating it, it becomes possible to anchor the organ in a plane. Here, anchoring the organ in a plane means that, for example, the organ is anchored by suturing such that the suture forms a triangular or quadrilateral surface within the organ. When the organ is thus anchored, there is no separation within the plane of the organ from the paries.

### BRIEF INTRODUCTION TO THE DRAWINGS

Embodiments of the present invention will now be described by way of example only with reference to the accompanying drawings in which:

FIG. 1 is a side view of the medical suturing device set pertaining to Embodiment 1 of the present invention.

FIG. 2 is an explanatory diagram of the suture and the insertion puncture needle of FIG. 1.

FIG. 3 is a side view of the essential elements of the extraction device of FIG. 1.

FIG. 4 is a diagram explaining the operation of Embodiment 1 of the present invention.

FIG. 5 is a diagram explaining the operation subsequent to FIG. 4.

FIG. 6 is a diagram explaining the operation subsequent to FIG. 5.

FIG. 7 is a diagram explaining the operation subsequent to FIG. 6.

FIG. 8 is a diagram explaining the operation subsequent to FIG. 7.

FIG. 9 is a diagram explaining the operation subsequent to FIG. 8.

FIG. 10 is a side view of the essential elements of the extraction device of the medical suturing device set pertaining to Embodiment 2 of the present invention.

FIG. 11 is a side view of the essential elements of the extraction device of the medical suturing device set pertaining to Embodiment 3 of the present invention.

FIG. 12 is a side view of the essential elements of the extraction device of the medical suturing device set pertaining to Embodiment 4 of the present invention.

FIG. 13 is a side view of the essential elements of the extraction device of the medical suturing device set pertaining to Embodiment 5 of the present invention.

FIG. 14 is a side view of the essential elements of the extraction device of the medical suturing device set pertaining to Embodiment 6 of the present invention.

### DETAILED DESCRIPTION OF THE INVENTION

### Embodiment 1

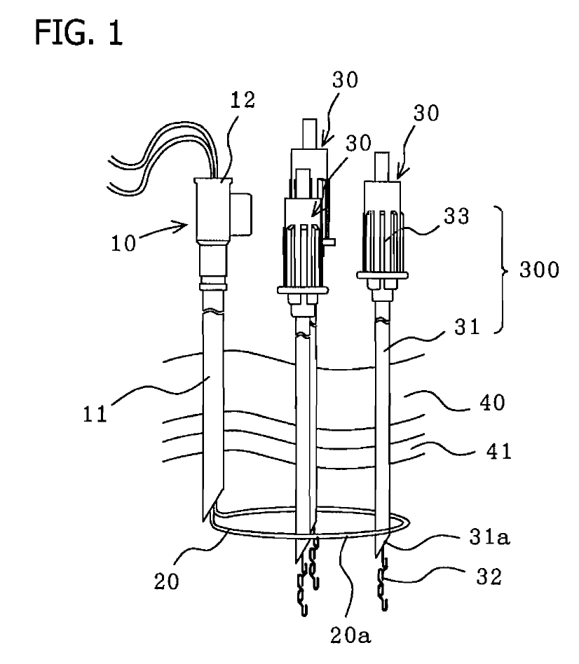

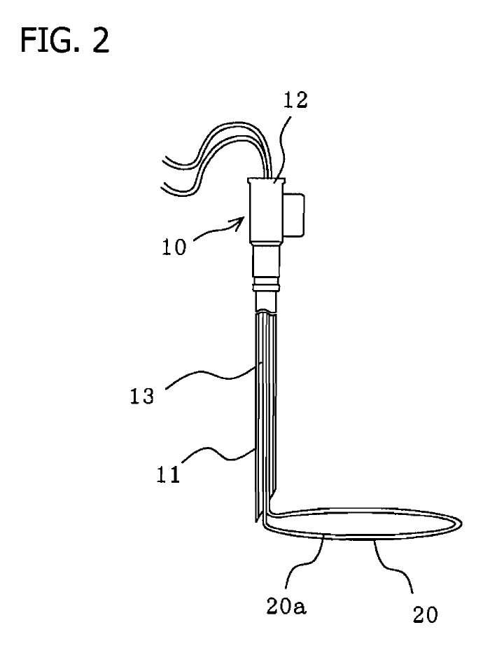

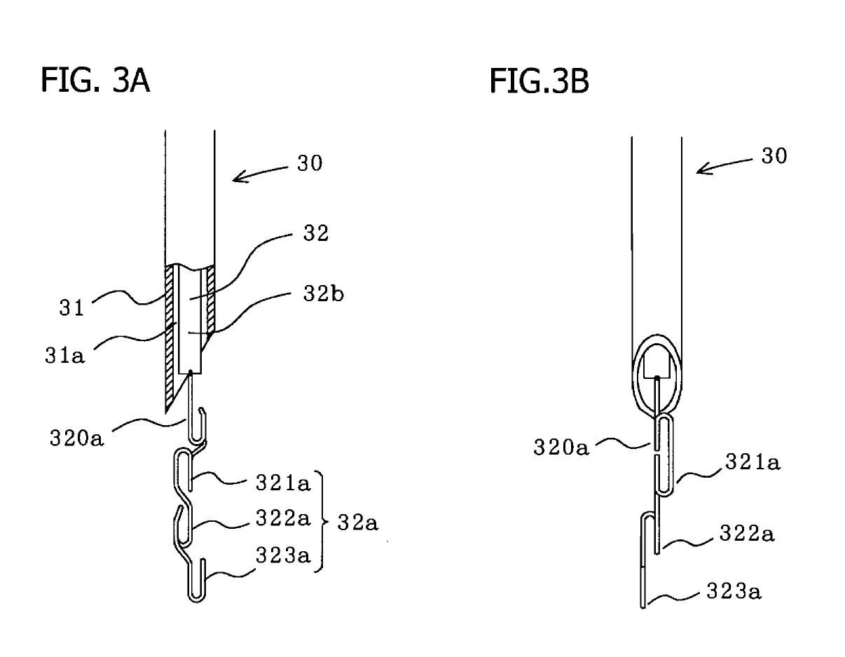

FIG. 1 is a side view of the medical suturing device set pertaining to Embodiment 1 of the present invention. FIG. 2 is a partially axially cut-away mode side view of the suture and insertion puncture needle of FIG. 1. FIG. 3 is a side view of the essential elements of the extraction device of FIG. 1. As shown in the figures, the medical suturing device set is structured by a insertion puncture needle 10 furnished with a suture 20, and several extraction devices 30 (3 in the illustrated example).

The insertion puncture needle 10 is structured using a puncture needle 11 and hub 12, with insertion through-hole 13 boring through these internally in the axial direction, such that suture 20 can slide within insertion through-hole 13. The puncture needle 11 is formed so that its apical tip has a sharp angle, and suture 20 protrudes from the apical tip so as to form a loop section 20a. The suture 20 may be constituted by a nylon thread, for example.

The extraction device 30 is formed by a extraction puncture needle 300, which has a hub 33 and a metal needle 31 having an apical tip formed into a sharp angle, and pulling out device 32. A cavity 31a is formed axially within extraction puncture needle 300. The extraction device 30 is structured such that pulling out device 32 can slide within cavity 31a and exit and enter through the apical tip of extraction puncture needle 300. The pulling out device 32 comprises a manipulation section 32b and an engagement member 32a that is contiguous with one end of the manipulation section 32b.

The engagement member 32a is an entity where multiple hook elements have been serially fixed in a sequential alignment (approximately linear): first hook element 320a, second hook element 321a, third hook element 322a, and fourth hook element 323a, with an end of first hook element 320a being connected to one end of manipulation section 32b.

The respective hook elements constitute hook shapes shaped approximately like the letter “J,” and are furnished with a base that can support part of suture 20, a primary extension that is connected to part of the base and extends towards the basal tip of pulling out device 32, a secondary extension that is connected to the other part of the base and that extends to the basal tip of pulling out device 32, and a gap that is formed so as to envelope the periphery of suture 20 by the base and the primary extension and the secondary extension. The linking of companion hook elements is formed by connections between the primary extension and secondary extension of adjacent hook elements.

In this case, the J-shaped flat surface of second hook element 321a is attached approximately perpendicular to the J-shaped flat surface of first hook element 320a, the J-shaped flat surface of third hook element 322a is attached approximately perpendicular to the J-shaped flat surface of second hook element 321a, and the J-shaped flat surface of fourth hook element 323a is attached approximately perpendicular to the J-shaped flat surface of third hook element 322a. It is also possible for them to be connected to each other at any angle, not necessarily perpendicular. When the adjacent hook elements 320a, 321a, 322a, and 323a are positioned so as to be approximately perpendicular, the branches (projectional elements) of hook elements 320a, 321a, 322a, and 323a face towards the outside, extending in 4 directions.

The engagement member 32a may also be structured so that the J-shaped flat surface of first hook element 320a, the J-shaped flat surface of second hook element 321a, the J-shaped flat surface of third hook element 322a, and the J-shaped flat surface of fourth hook element 323a are positioned to be approximately on the same plane.

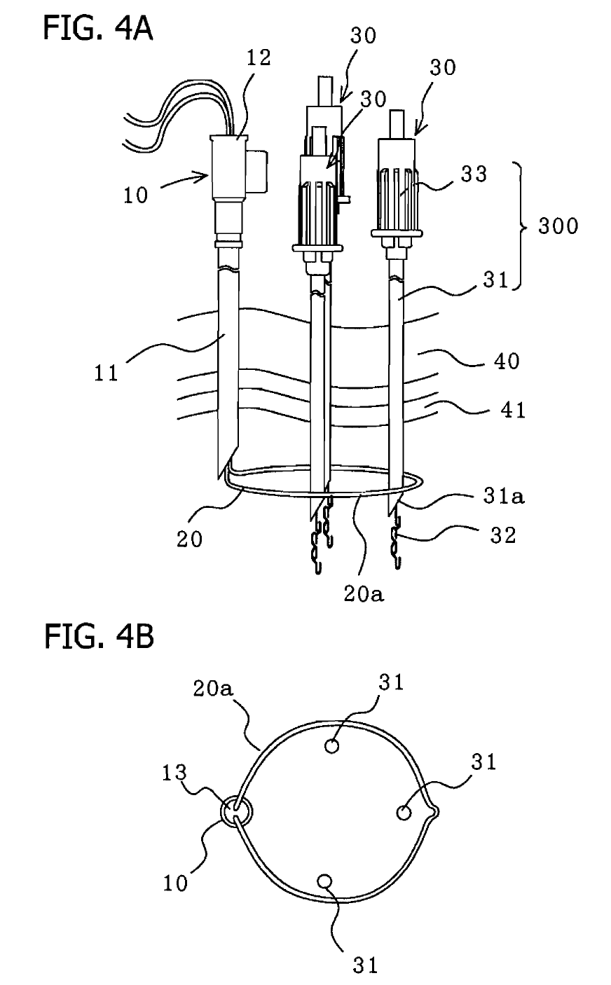

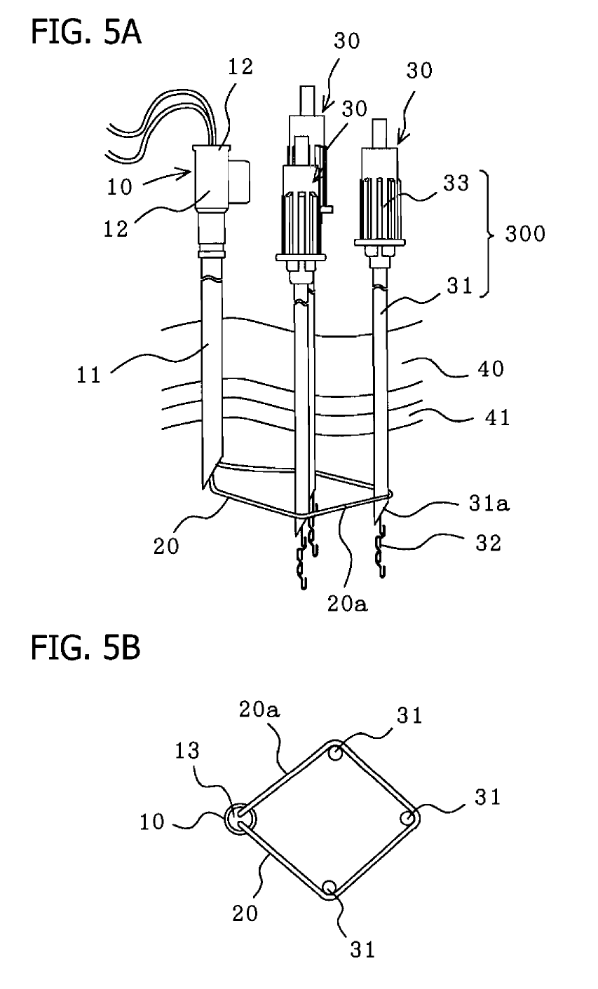

The method for using the medical suturing device set having the aforementioned structure (i.e., its operation), is explained in FIGS. 4-9. As shown in FIG. 4, the hub 12 of insertion puncture needle 10 is grasped, and the gastric wall 41 is punctured from abdominal wall 40 by puncture needle 11. After the puncture, suture 20 is caused to slide along insertion through-hole 13 of insertion puncture needle 10, and suture 20 is caused to project from the apical tip of insertion through-hole 13, thus forming loop section 20a. Then there are multiple punctures of extraction device 30 within the loop section 20a formed by suture 20. The pulling out device 32 is thrust from the tip of metal needle 31 of extraction device 30, and suture 20 is drawn from the outside of hub 12 of insertion puncture needle 10. When this is done, as shown in FIG. 5, loop section 20a is snugly attached to metal needle 31 of multiple extraction devices 30.

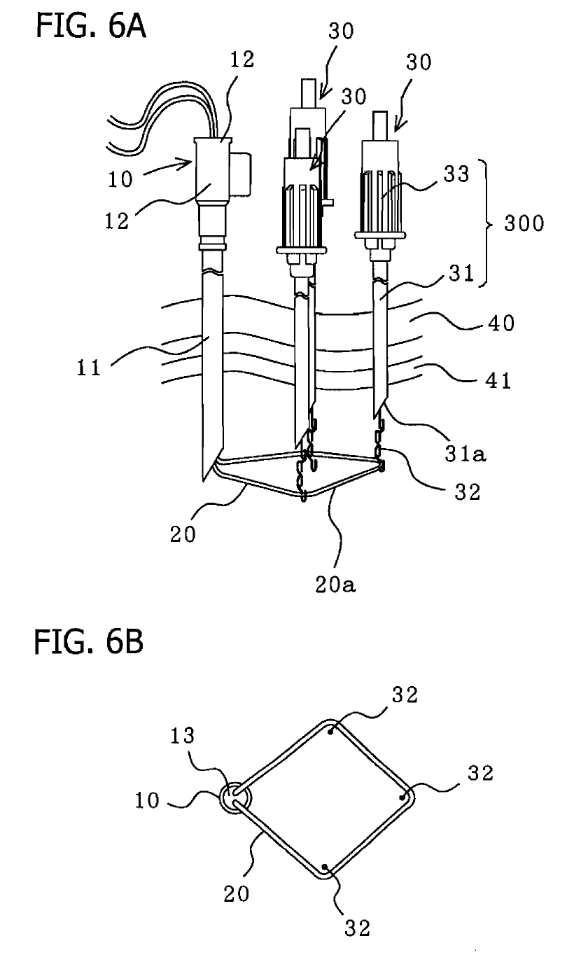

Next, as shown in FIG. 6, extraction device 30 is slightly drawn up to engage the loop section 20a of suture 20 in engagement member 32a of pulling out device 32. Then, in the engaged condition, pulling out device 32 is stowed in cavity 31a of metal needle 31, and in this condition extraction device 30 is pulled upwards, and the upwardly pulled suture 20 is separated from extraction device 30. Then insertion puncture needle 10 is removed. When loop section 20a is pulled out by pulling out device 32, it may be removed from the organism without being temporarily stowed in the cavity of extraction puncture needle 300; pulling out device 32 may be removed from suture 20, and then extraction puncture needle 300 and insertion puncture needle 10 may be removed.

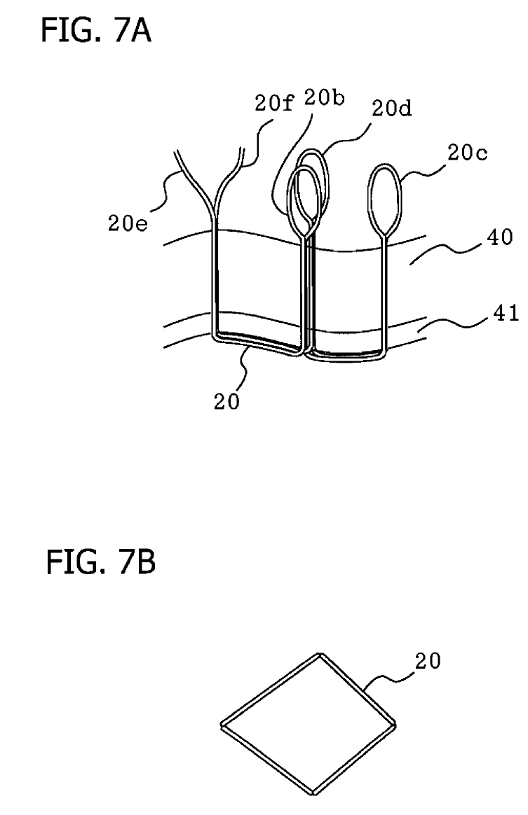

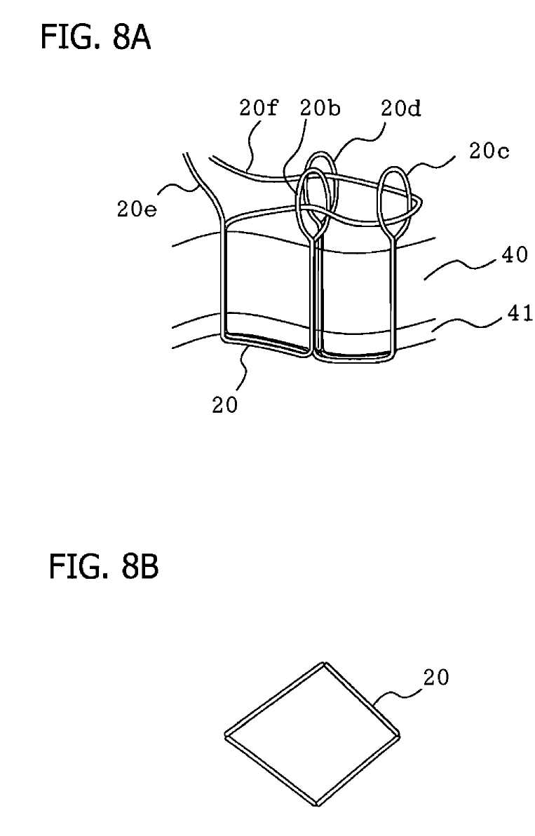

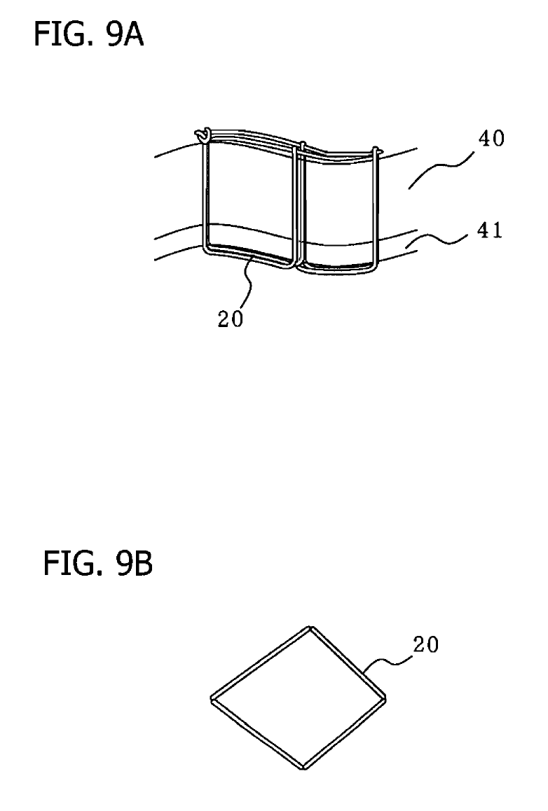

Then, as shown in FIG. 7, a number of thread circles 20b, 20c, and 20d, equal to the number of puncturing extraction devices 30, are formed outside the body. Then, as shown in FIG. 8, 20f, which is one of the suture ends positioned on the side of insertion puncture needle 10, is sequentially inserted into thread circles 20b, 20c, and 20d, which are positioned on the side of extraction device 30. Then, as shown in FIG. 9, suture tips 20e and 20f are drawn together, knotted in the desired tightness, and thus fixed.

The foregoing explanation described a case where insertion puncture needle 10 was inserted and loop section 20a was formed, and extraction device 30 was inserted within loop section 20a, but it is also possible for extraction device 30 to first be inserted, then insert insertion puncture needle 10 and form loop section 20a, draw up insertion puncture needle 10, and position extraction device 30 within loop section 20a. The case was explained where the stomach was the organ to be sutured, but also applies to cases where other organs, such as the gallbladder or kidney, for example, are sutured.

In Embodiment 1, the structure is such that insertion puncture needle 10 and extraction device 30 are separated, so it is possible to pull out suture 20 formed in a ring shape through insertion through-hole 13 of insertion puncture needle 10 by means of pulling out device 32 of extraction device 30 at a position that is different from the insertion position, which makes it possible to freely select the puncture site together with the patient, and which also makes it possible to anchor the organ in plane shape, positioned within the organism, with only a single suture. Also, pulling out device 32 exits and enters along cavity 31a of metal needle 31 of extraction device 30, which makes it possible to reduce the invasiveness towards the body when suture 20 is drawn out of the organism. This makes it possible to securely anchor the desired organ to the paries with low invasiveness, even by inexperienced health care personnel.

### Embodiment 2

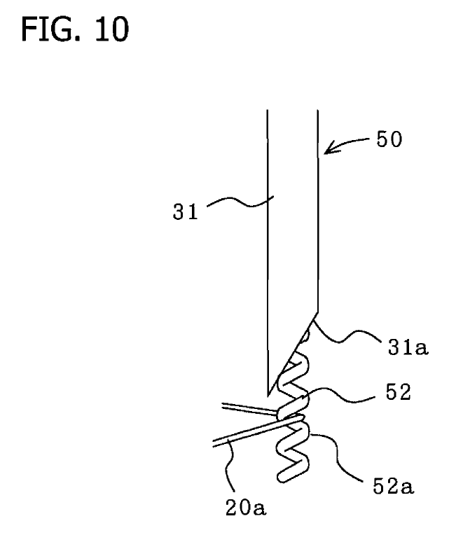

FIG. 10 is a side view of the essential elements of the extraction device pertaining to Embodiment 2 of the present invention. In Embodiment 2, engagement member 52a of pulling out device 52 has a helical shape, so as to seize loop section 20a of suture 20 on helical engagement member 52a. More specifically, the engagement member 52a of pulling out device 52 is structured by a continuous helical element, and this helical section thrusts out from insertion puncture needle 10 so as to engage part of the formed loop section 20a.

According to Embodiment 2, the engagement member 52a of pulling out device 52 is formed as a helical shape, so without being limited to the circumferential position of pulling out device 52, it is easily able to engage the suture that is disposed in annular shape. Other than pulling out device 52, the structure, method of use, and results of Embodiment 2 are identical to those of Embodiment 1 so the explanation [of identical aspects] may be omitted.

### Embodiment 3

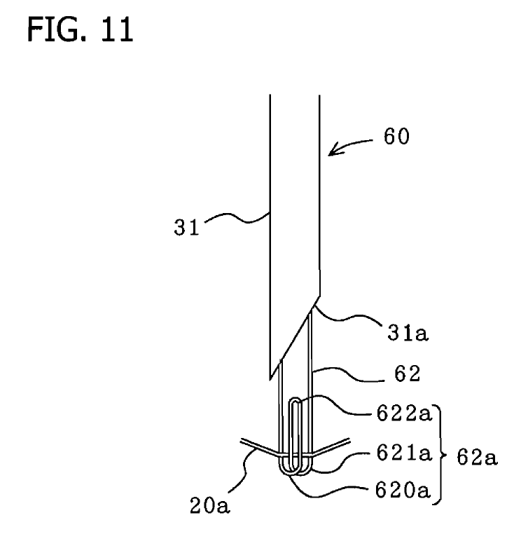

FIG. 11 is a side view of the essential elements of the extraction device pertaining to Embodiment 3 of the present invention. In Embodiment 3, engagement member 62a of pulling out device 62 has a paperclip shape, so that loop section 20a of suture 20 is seized on the paperclip-shaped section.

Here, pulling out device 62 is structured by a paperclip-shaped engagement section; for example, it may be formed by 3 U-shaped sections. More specifically, as shown in FIG. 11, a linear element has been bent on the side of metal needle 31 to form primary U-shaped element 620a; the vicinity of primary U-shaped element 620a has been bent on the side of primary U-shaped element 620a to form tertiary U-shaped element 622a; the vicinity of primary U-shaped element 620a has been further bent on the side of metal needle 31 to form secondary U-shaped element 621a; and the primary and secondary U-shaped elements 620a and 621a positioned in identical orientations with partial overlap and tertiary U-shaped element 622a positioned in the opposite orientation together form pulling out device 62.

In Embodiment 3, the tip of pulling out device 62 is able to seize loop section 20a of suture 20. Otherwise, the structure, method of use, and results of Embodiment 3 are identical to those of Embodiments 1 and 2, with the exception that the circumferential orientation has been limited, so the explanation may be omitted.

### Embodiment 4

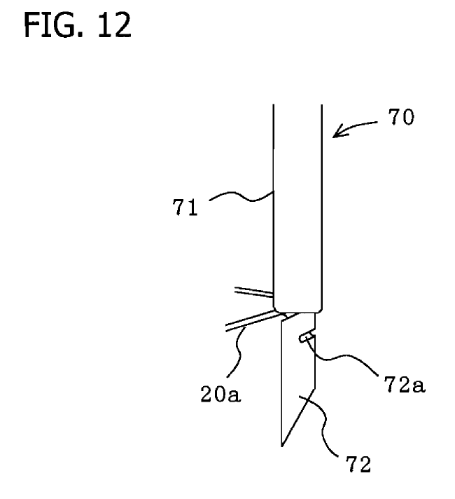

FIG. 12 is a side view of the essential elements of the extraction device pertaining to Embodiment 4 of the present invention. In Embodiment 4, extraction device 70 is structured from mantle 71 and extraction needle 72, and a helical groove 72a is furnished on the lateral surface of extraction needle 72.

The extraction device 70 is structured by mantle 71, hub 33, and extraction needle 72, and an internal cavity (not shown) is formed axially within mantle 71. The mantle 71 is formed so that its front tip is approximately perpendicular to its axis, thus differing from metal needle 31 shown in Embodiments 1-3, which is formed into a sharp angle. The extraction needle 72 is structured so that it is able to slide within the cavity and enter and exit from the apical tip. The extraction needle 72 is structured by a rod member, the apical tip of which forms a sharp angle, which is furnished with helical groove 72a axially along the outer lateral surface of the rod member, such that the loop section 20a of suture 20 can be seized in helical groove 72a.

The extraction device 70 having the structure described above is used as follows: when extraction device 70 is inserted, the tip of extraction needle 72 punctures the organism in a condition where the tip of extraction needle 72 is exposed from the tip of mantle 71. At this time, helical groove 72a is stowed in the cavity of mantle 71. Then, after puncturing to a predetermined depth, the extraction needle 72 is inserted even deeper, such that a portion on which helical groove 72a has been formed is sufficiently exposed. In this condition it engages loop section 20a of suture 20. Then the apical tip of extraction needle 72 is stowed within the cavity of mantle 71, and in this condition extraction device 70 is drawn up, and after the drawing up operation suture 20 is removed from extraction device 70.

Embodiment 4 makes it possible to engage loop section 20a of suture 20 by helical groove 72a continuously furnished on extraction needle 72. In other respects the structure, method of use, and results are identical to that of Embodiments 1 and 2, so their explanation is omitted.

### Embodiment 5

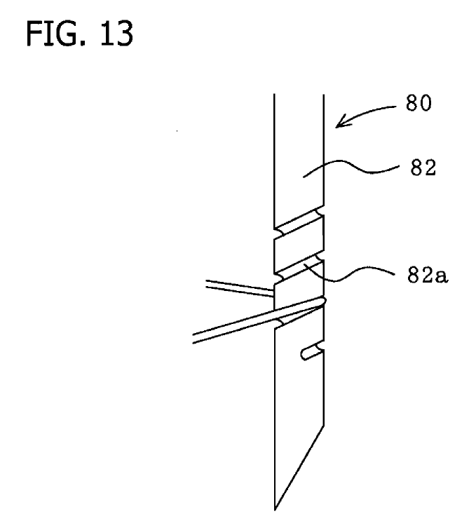

FIG. 13 is a side view of the essential elements of the extraction device pertaining to Embodiment 5 of the present invention. In Embodiment 4, the extraction device was structured by an extraction needle and an outer mantle, but in Embodiment 5 extraction device 80 is structured from only a hub 33 and extraction needle 82. The extraction needle 82 is structured by a rod member, the apical tip of which is formed into a sharp angle, and helical groove 82a is furnished axially on the outer lateral surface of the rod member, such that loop section 20a of suture 20 may be seized in helical groove 82a.

The aforementioned extraction device 80 punctures the organism directly by extraction needle 82, the loop section 20a of suture 20 is caught in helical groove 82a of extraction needle 82, and then drawn out of the body.

Embodiment 5 makes it possible to seize loop section 20a of suture 20 along the axis of extraction needle 82 by the continuous helical groove 82a, and then draw suture 20 out of the body. In other respects the structure, method of use, and results are identical to that of Embodiment 4, so their explanation is omitted.

### Embodiment 6

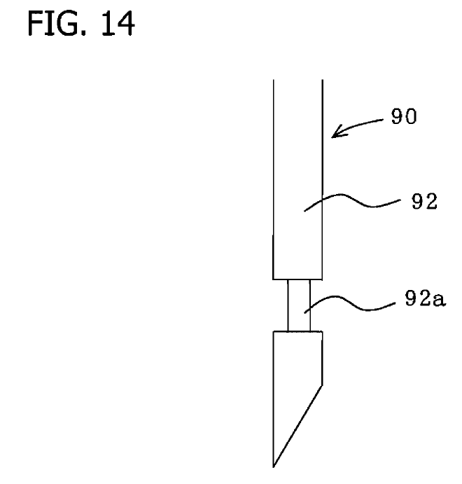

FIG. 14 is a side view of the essential elements for Embodiment 6 of the present invention. Embodiment 6 is similar to Embodiment 5 in that extraction device 90 is structured by only a hub 33 and extraction needle 92.

The extraction needle 92 is structured by a rod member, the apical tip of which is formed into a sharp angle, and concavity 92a having a fixed width axially, and which is radially reduced around the perimeter of said rod member, such that loop section 20a of suture 20 is seized in this concavity 92a.

Embodiment 6 makes it possible to seize loop section 20a of suture 20 by a fixed width along the axis of concavity 92a furnished on extraction needle 92, and then draw suture 20 out of the body. In other respects the structure, method of use, and results are identical to that of Embodiment 5, so their explanation is omitted.

1. A surgical suture withdrawal device for use in extracting a suture loop from within an organism to the outside of the organism comprising:
a hollow needle and
an engagement member movable within the needle and capable of catching onto a suture positioned within the organism and drawing at least part of the suture outside of the organism.

2. The device according to claim 1, wherein the engagement member may pass entirely through the hollow needle.

3. The device according to claim 1 wherein the engagement member comprises at least one hook element.

4. The device according to claim 1 wherein the engagement member comprises a helical engagement section.

5. The device according to claim 1 wherein the engagement member comprises a rod member having at least one concave section.

6. A surgical suturing set comprising one or more surgical suture withdrawal devices, each device comprising:
a hollow needle and
an engagement member movable within the needle and capable of catching onto a suture positioned within the organism and drawing at least part of the suture outside of the organism and a suture loop insertion device for introducing a suture loop into said organism.

7. The surgical suturing set according to claim 6, wherein the set comprises three suture withdrawal devices.
